(19) United States
(12) Patent Application Publication
(10) Pub. No.: US 2025/0009507 A1
(43) Pub. Date: Jan. 9, 2025

(54) GUIDE CATHETER FOR AN IMPLANT DELIVERY APPARATUS

(71) Applicant: EDWARDS LIFESCIENCES CORPORATION, Irvine, CA (US)

(72) Inventors: Kevin Jiang, Irvine, CA (US); Darshin S. Patel, Lake Forest, CA (US); Kurt Kelly Reed, Costa Mesa, CA (US)

(21) Appl. No.: 18/885,198

(22) Filed: Sep. 13, 2024

Related U.S. Application Data
(63) Continuation of application No. PCT/US2023/015342, filed on Mar. 16, 2023.
(60) Provisional application No. 63/269,522, filed on Mar. 17, 2022.

(51) Int. Cl. A61F 2/24

(52) U.S. Cl. CPC A61F 2/2436

(57) ABSTRACT

Devices and methods for providing a reservoir of fluid within a handle of a guide catheter are disclosed. As one example, a delivery apparatus can include a handle including a proximal segment including one or more fluid seals mounted within the proximal segment, an intermediate segment disposed adjacent and distal to the proximal segment, the intermediate segment including an inner surface defining a first lumen having a first inner diameter, and a distal segment disposed adjacent and distal to the intermediate segment. The delivery apparatus can further include a shaft extending distally from the handle, the shaft including a second lumen extending between a distal end and a proximal end of the shaft, the second lumen including a second inner diameter that is smaller than the first inner diameter of the first lumen.

### CROSS-REFERENCE TO RELATED APPLICATIONS

This application is a continuation of PCT Patent Application No. PCT/US2023/015342, filed on Mar. 16, 2023, which claims the benefit of U.S. Provisional Application No. 63/269,522, filed Mar. 17, 2022, each of these applications being incorporated by reference herein in their entireties.

### FIELD

The present disclosure relates to guide catheters for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing a delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The delivery apparatus can be pushed through a main lumen of the guide catheter in order to help navigate the delivery apparatus to a target implantation site within the patient.

### SUMMARY

Described herein are prosthetic heart valves, delivery apparatuses, guide catheters, and methods for implanting prosthetic heart valves. The disclosed guide catheters can, for example, be configured to receive a portion of a delivery apparatus within a main lumen of the guide catheter in order to introduce the delivery apparatus into a patient's vasculature. In some examples, the guide catheter can include a reservoir of fluid that is fluidly coupled with the main lumen within a handle of the guide catheter that provides a location for fluid (e.g., air) to accumulate for removal as a delivery apparatus is being navigated through the main lumen. As such, the devices and methods disclosed herein can, among other things, overcome one or more of the deficiencies of typical guide catheters.

In one representative example, a delivery apparatus comprises a handle comprising: a proximal segment including one or more fluid seals mounted within the proximal segment, the fluid seals configured to allow insertion of a device into the handle and prevent fluid flow past the fluid seals; an intermediate segment disposed adjacent and distal to the proximal segment, the intermediate segment including an inner surface defining a first lumen, wherein the first lumen has a first inner diameter, wherein the first lumen includes an inlet and an outlet; and a distal segment disposed adjacent and distal to the intermediate segment; and a shaft extending distally from the handle, the shaft including a distal end, a proximal end, and a second lumen extending between the distal end and the proximal end of the shaft, wherein the proximal end of the shaft is disposed within the distal segment of the handle and is coupled to the outlet of the first lumen, wherein the second lumen includes a second inner diameter that is smaller than the first inner diameter of the first lumen.

In another representative example, a delivery apparatus comprises a seal housing assembly including a plurality of fluid seals, the plurality of fluid seals configured to allow insertion of a device into the delivery apparatus and prevent fluid flow past the fluid seals; a reservoir coupled to and extending distally from the seal housing assembly, the reservoir including a reservoir lumen having a first inner diameter and a flush lumen in fluid communication with the reservoir lumen, wherein the reservoir lumen includes an inlet coupled to the seal housing assembly and an outlet; and a shaft coupled to and extending distally from the outlet of the reservoir, the shaft including a shaft lumen in fluid communication with the reservoir lumen, and wherein the shaft lumen includes a second inner diameter that is smaller than the first inner diameter of the reservoir lumen.

In another representative example, a method for implanting a prosthetic medical device comprises inserting a shaft of a guide catheter into a vessel of a patient; inserting a distal end portion of a first implant catheter into a proximal end of a handle of the guide catheter and pushing the distal end portion of the first implant catheter through a reservoir of the handle of the guide catheter and then through a main lumen of the shaft of the guide catheter toward a target implantation site for a prosthetic medical device mounted on the distal end portion of the first implant catheter, the reservoir having an inner diameter that is larger than an inner diameter of the main lumen; and after inserting the distal end portion of the first implant catheter, removing fluid and/or air out of the reservoir through a tube that is fluidly coupled with the reservoir via a port of the reservoir.

In another representative example, a delivery apparatus comprises a shaft including a first lumen having a first inner diameter; and a handle including an air trap portion connected to a proximal end of the shaft and a seal stack portion adjacent and proximal to the air trap portion, the air trap portion including a second lumen coaxial with the first lumen, the second lumen having a second inner diameter that is larger than the first inner diameter, wherein the seal stack portion includes one or more fluid seals mounted within the seal stack portion and configured to allow insertion of a device therethrough.

In another representative example, a delivery assembly comprises an implant catheter; and a guide catheter comprising: a shaft having a distal end and a proximal end, the shaft including a main lumen configured to receive a portion of the implant catheter therethrough, the main lumen having a first inner diameter; and a handle including a reservoir coupled to the proximal end of the shaft and a seal housing assembly adjacent and proximal to the reservoir, the reservoir including a reservoir lumen in fluid communication with the main lumen, the reservoir lumen having a second inner diameter that is larger than the first inner diameter, wherein the seal housing assembly includes one or more fluid seals mounted within the seal housing assembly and configured to allow insertion of the implant catheter therethrough.

In another representative example, a delivery apparatus comprises a handle having a proximal end and a distal end, the handle comprising: a seal housing assembly at the proximal end of the handle, the seal housing assembly including one or more fluid seals mounted within the seal housing assembly, the fluid seals configured to allow insertion of a device into the handle and prevent fluid flow past the fluid seals; an outer housing at the distal end of the handle; and a reservoir having an inlet and an outlet, the inlet coupled to the seal housing assembly and the outlet coupled to the outer housing, the reservoir including a first lumen extending from the inlet to the outlet, the first lumen having a first inner diameter; and a shaft having a proximal end and a distal end, wherein the proximal end of the shaft is disposed within the outer housing and is coupled to the outlet of the reservoir, wherein the second lumen includes a second inner diameter that is smaller than the first inner diameter of the first lumen.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side view of a guide catheter configured to receive a delivery apparatus and guide the delivery apparatus through a portion of a patient's vasculature, according to one example.

FIG. 6 is a cross-sectional side view of the guide catheter of FIG. 5.

FIG. 7 is a side view of a delivery apparatus for a docking device, according to one example.

FIG. 8 is a perspective view of a docking device for use with the delivery apparatus of FIG. 7, according to one example.

FIG. 9 is a perspective view of a delivery apparatus for a prosthetic heart valve, according to one example.

FIG. 10 is a perspective view of a prosthetic heart valve for use with the delivery apparatus of FIG. 9, according to one example.

FIG. 11A is a cross-sectional side view of a guide catheter, according to another example.

FIG. 11B is a cross-sectional side view of a guide catheter, according to yet another example.

FIG. 12 is a perspective view of a reservoir for a guide catheter, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Introduction to the Disclosed Technology

Described herein are various systems, apparatuses, methods, or the like, that, in some examples, can be used in or with delivery apparatuses for prosthetic medical devices (such as prosthetic heart valves or docking devices). In some examples, such systems, apparatuses, and/or methods can provide a reservoir of fluid within a handle of a delivery apparatus that provides a location for fluid (e.g., air) to accumulate for removal as a prosthetic medical device mounted on another delivery apparatus is navigated through the reservoir and a lumen of the delivery apparatus toward an implantation site in a body of a patient.

For example, the delivery apparatus can be inserted into a vessel of a patient and another delivery apparatus including a prosthetic medical device (e.g., a prosthetic heart valve) mounted thereon can be navigated through a main lumen of the delivery apparatus toward a target implantation site for the prosthetic medical device. In some examples, the delivery apparatus can include a reservoir within a handle of the delivery apparatus that has a larger inner diameter than an inner diameter of the main lumen. As such, the reservoir can provide a location (e.g., at the larger inner diameter) for air to accumulate and/or become trapped while another delivery apparatus is navigated through the delivery apparatus. The trapped air can be removed from the system, thereby preventing or reducing the risk of air entering the patient's vasculature.

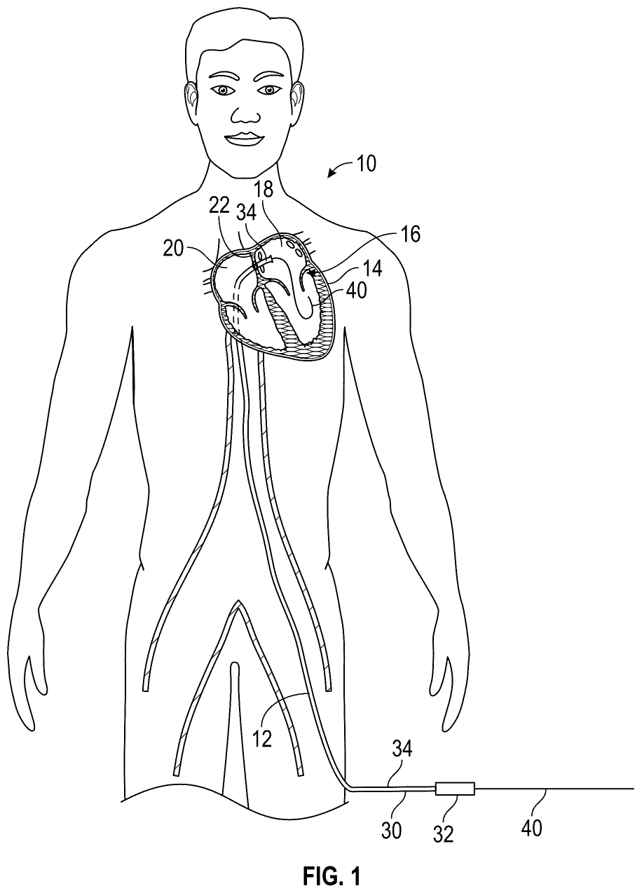

In some examples, the delivery apparatuses disclosed herein can be used to introduce one or more delivery apparatuses (or implant catheters) into the vasculature of a patient and guide the one or more delivery apparatuses at least partially through the vasculature toward a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus and then the prosthetic heart valve delivery apparatus is used to deliver a transcatheter prosthetic heart valve inside the docking device.

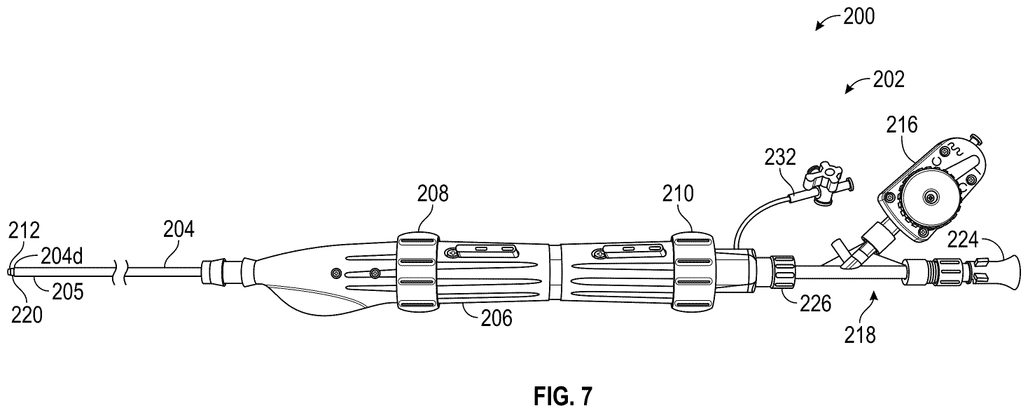

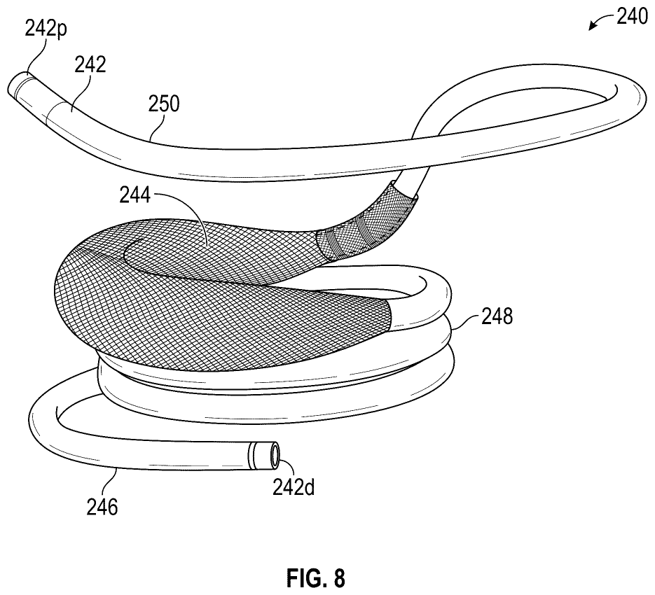

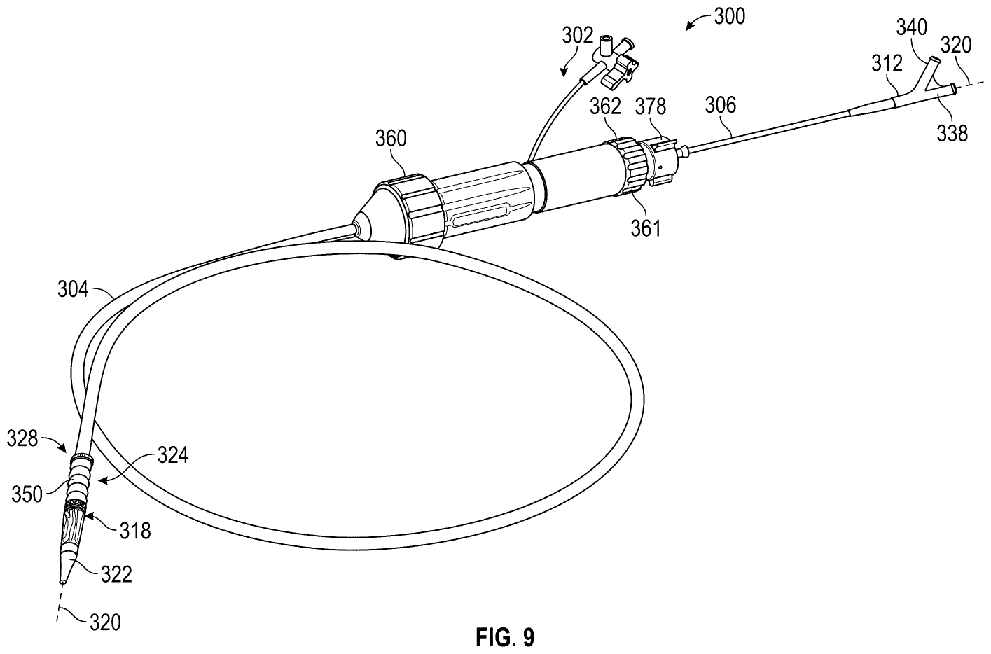

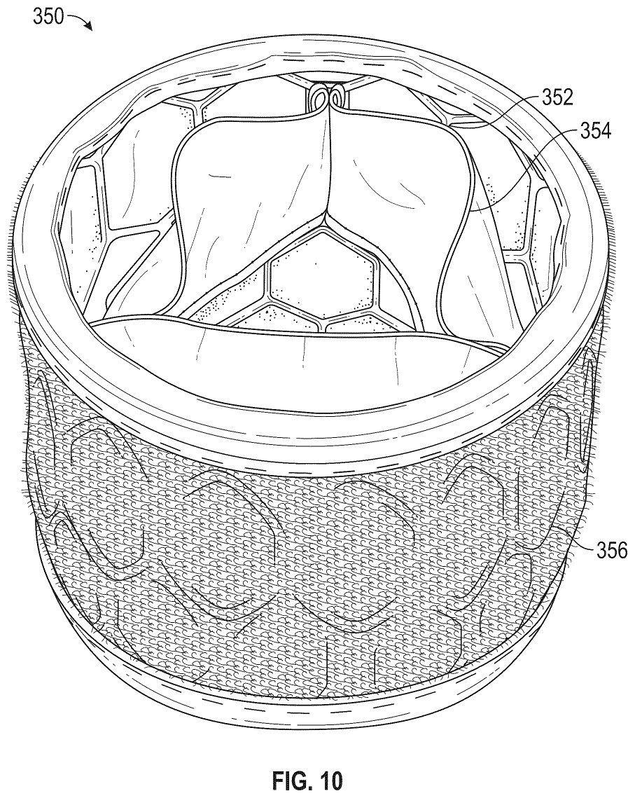

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary delivery apparatus for delivering a docking device at a native heart valve is shown in FIG. 7, and an exemplary docking device is shown in FIG. 8. An exemplary delivery apparatus for delivering a prosthetic heart valve within a docking device at a native heart valve is shown in FIG. 9, and an exemplary prosthetic heart valve is shown in FIG. 10.

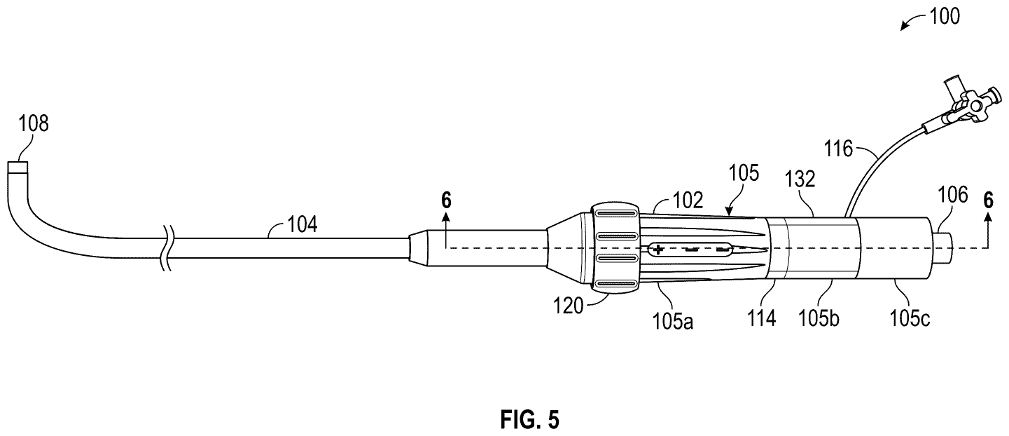

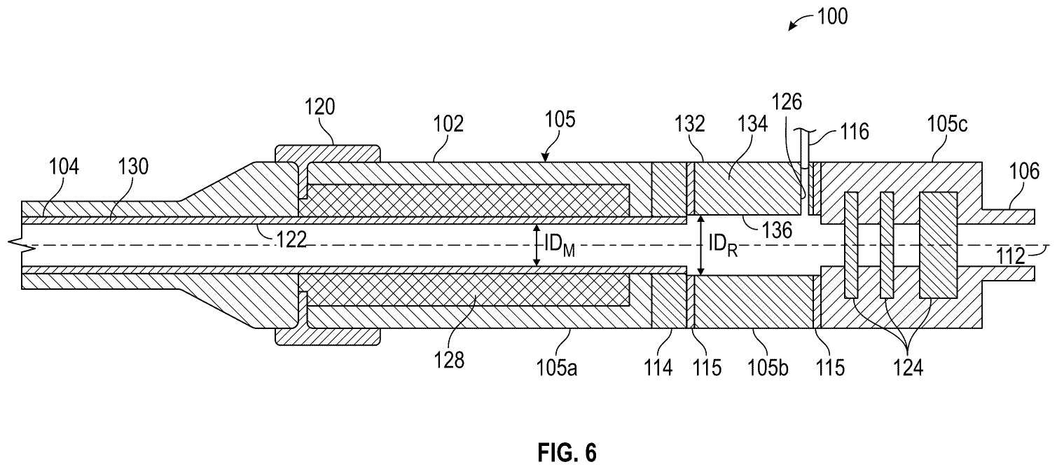

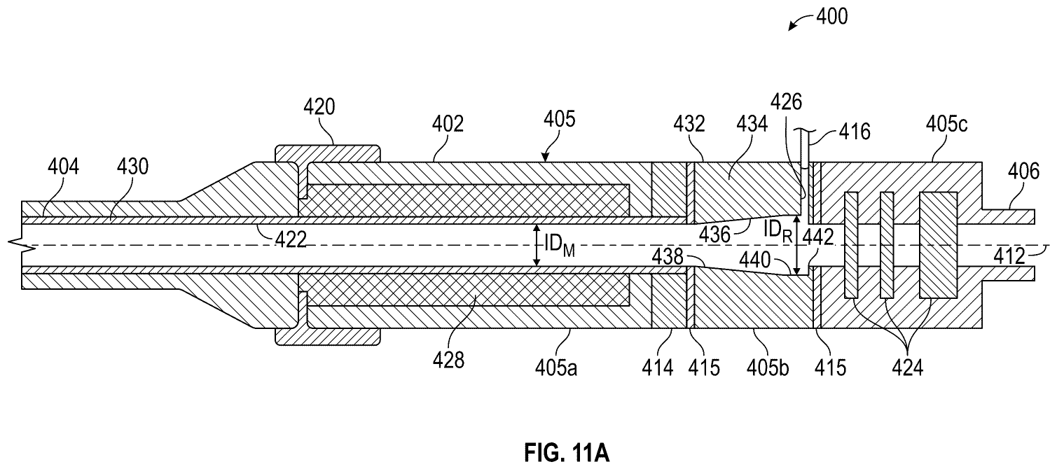

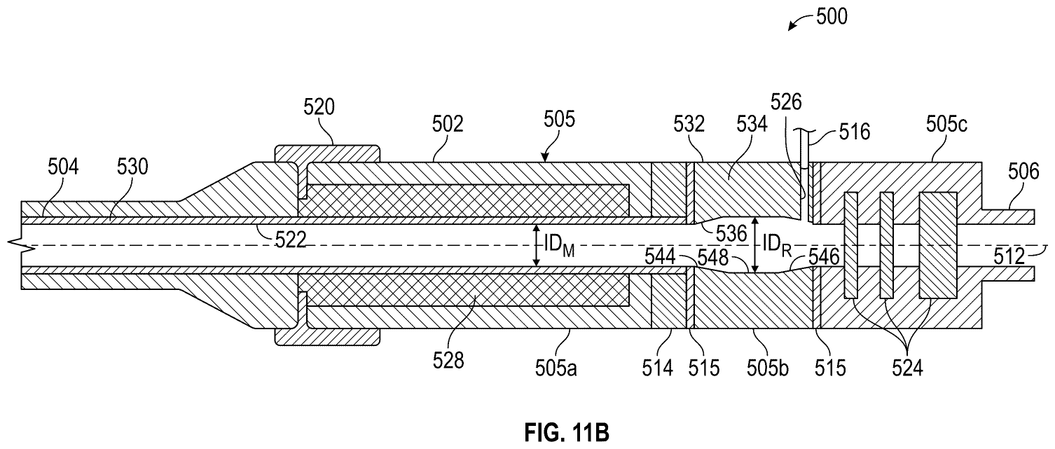

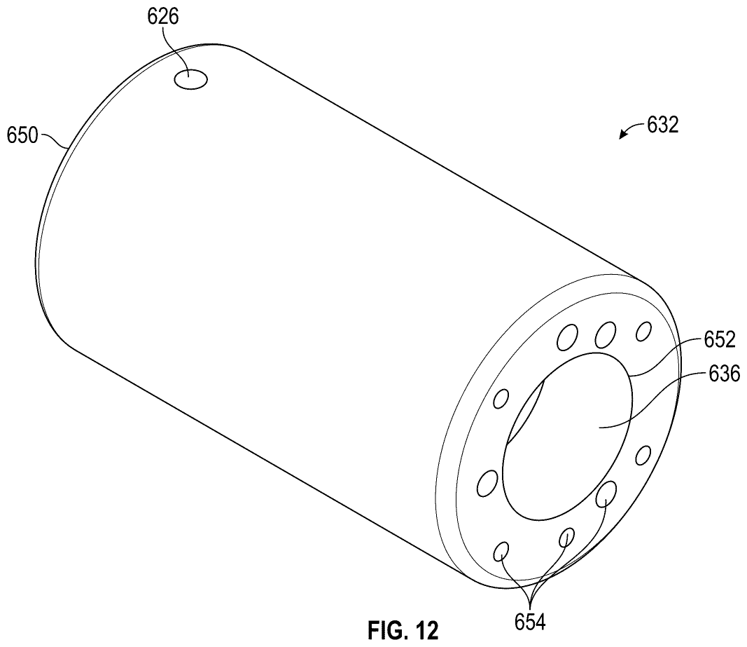

Exemplary guide catheters are shown in more detail in FIGS. 5-6 and 11A-11B. In some examples, as shown in FIGS. 6 and 11A-11B, a guide catheter can include a reservoir within a handle of the guide catheter that is filled with fluid and fluidly coupled to the main lumen of the guide catheter. Additional details of an exemplary reservoir are shown in FIG. 12. The reservoir can provide a location for air to collect and/or become trapped within the reservoir (e.g., a location having a larger inner diameter than the main lumen of the guide catheter, etc.), where such air may have been introduced into the system as a delivery apparatus is introduced through the guide catheter. Subsequently, in some instances, the trapped air may be removed from the reservoir (e.g., via a port). As a result, the reservoir prevents or reduces the likelihood of air from moving distally through the system.

### Examples of the Disclosed Technology

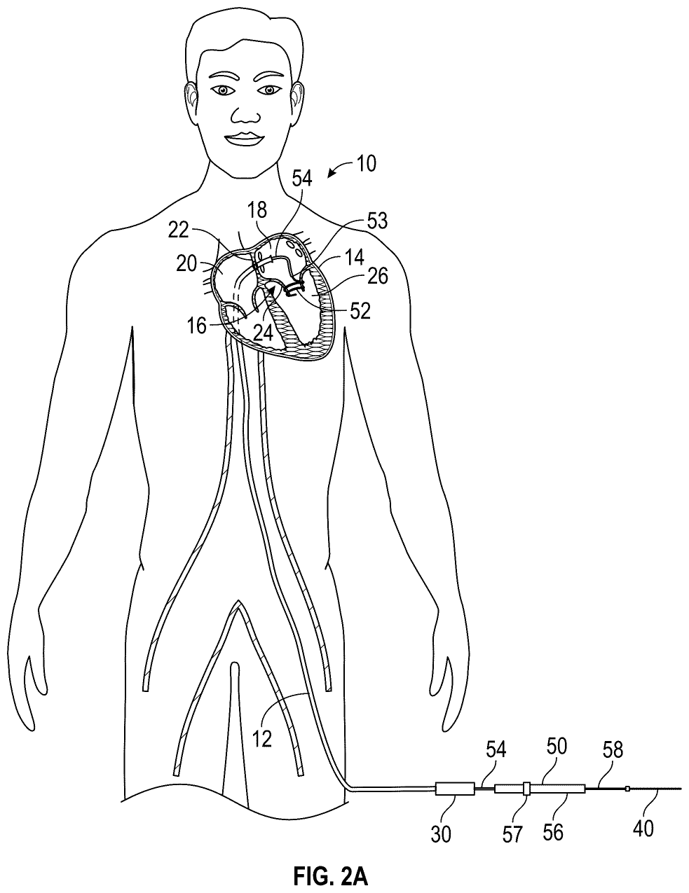

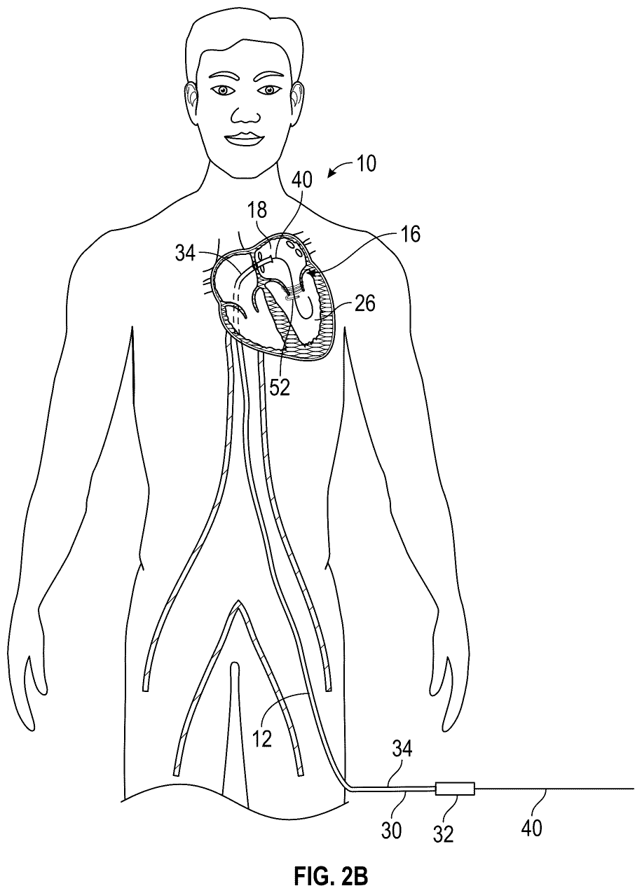

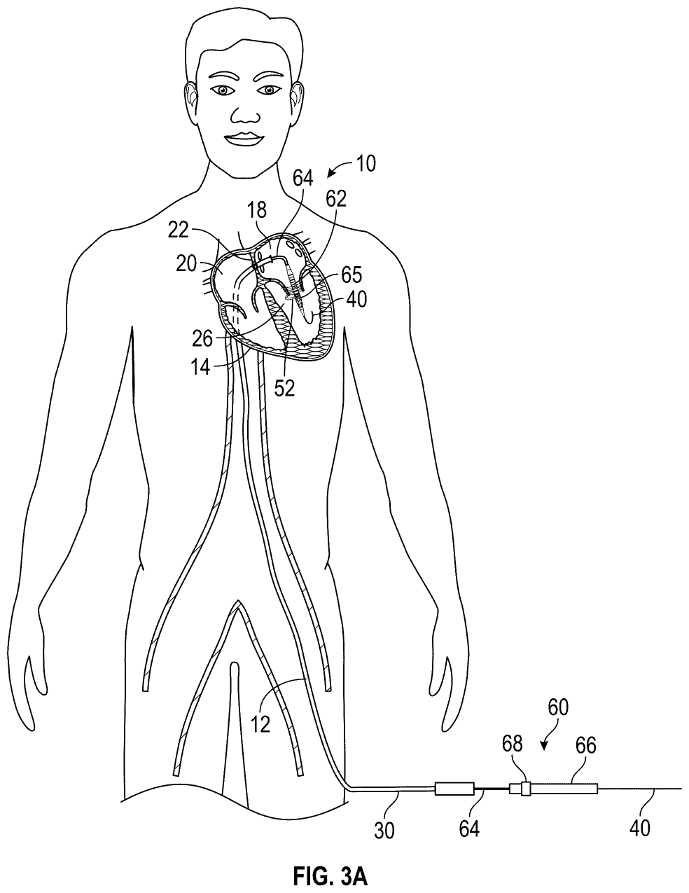

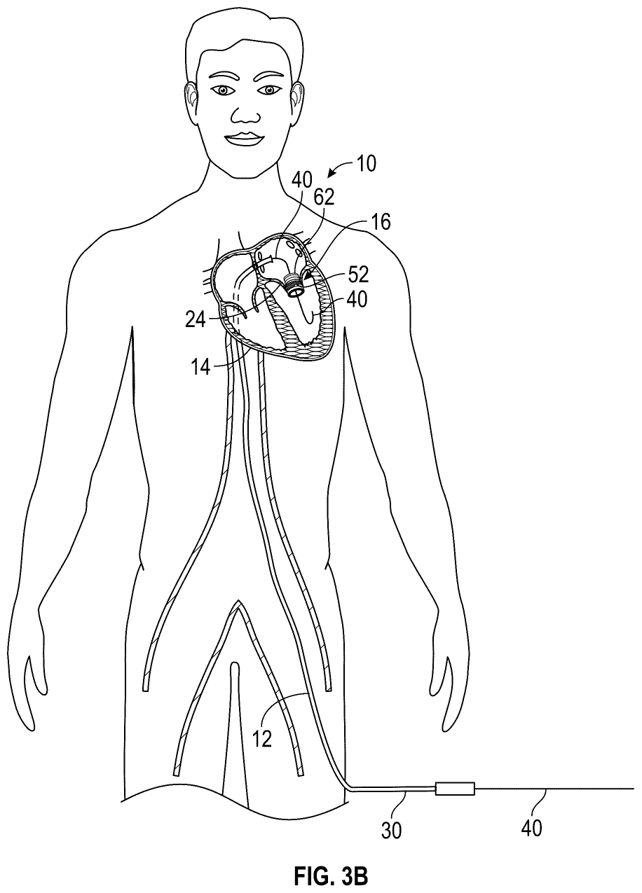

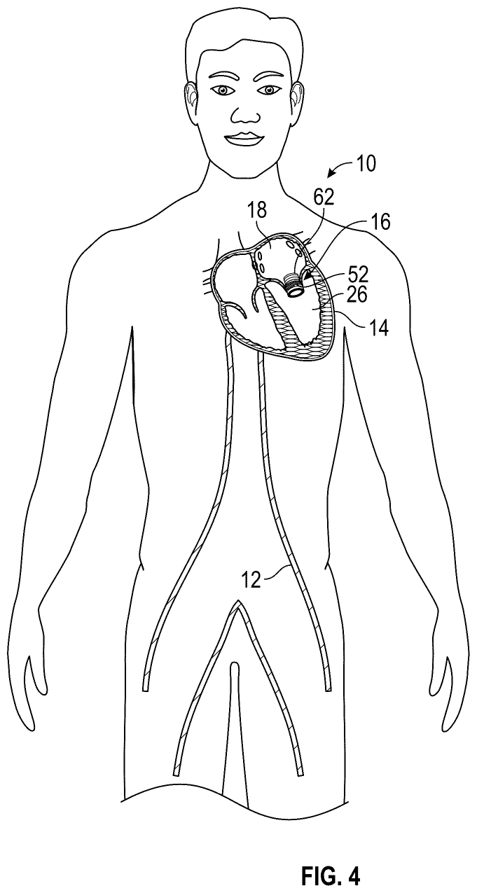

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

FIGS. 5 and 6 illustrate a guide catheter, which is referred to below as a guide sheath 100 (but can also be referred to herein as a “delivery apparatus” or an “introducer device”), according to one example. In some examples, the guide sheath 100 can be used as the guide catheter 30 in a prosthetic valve implantation procedure, as described above with reference to FIGS. 1-4. The guide sheath 100 can be configured to be inserted into a patient's vasculature and receive an implant catheter (and/or other delivery apparatus) therein in order to introduce the implant catheter into the patient's vasculature and at least partially guide the implant catheter to a target implantation site. Examples of implant catheters for prosthetic medical devices (referred to below as “delivery apparatus 200” and “delivery apparatus 300”) that can be received within the guide sheath 100 are shown in FIGS. 7 and 9 respectively, as described further below. Though the guide sheath 100 is described herein as being used with the delivery apparatus 200 and the delivery apparatus 300, the guide sheath 100 can be configured to receive a variety of delivery apparatuses or implant catheters, such as alternate docking device delivery apparatuses, alternate prosthetic heart valve delivery apparatuses, and/or delivery apparatuses for other medical devices or medical therapies.

Referring still to FIGS. 5-6, the guide sheath 100 comprises a handle 102, an elongated shaft 104 extending distally from the handle 102, and a central longitudinal axis 112. The shaft 104 has a main (or primary) lumen 122 that is defined by an inner surface of a wall 130 of the shaft 104. The main lumen 122 is configured to receive a delivery apparatus therein (such as any of the prosthetic device delivery apparatuses or implant catheters described herein). In some examples, as shown in FIG. 6, the shaft 104 can extend into the handle 102. Further, in some examples, the main lumen 122 can extend through the handle 102 to an inlet port 106 disposed at a proximal end of the handle 102. Thus, in some examples, an inner surface of a wall of a portion of the handle (e.g., at the proximal end) can further define the main lumen 122. Thus, the main lumen 122 can extend from the inlet port 106 to a distal end 108 of the shaft 104.

The handle 102 can include a housing 105. Within the housing 105, the handle 102 can include one or more seals 124 and a reservoir 132. In some instances, the housing 105 can be integrally formed as a single, unitary component. In other instances, as depicted, the housing 105 can comprise one or more segments that are formed as separate components that are coupled together (e.g., via fasteners, adhesive, mating features, and/or other means for coupling). For example, the housing 105 can comprise a main segment 105a, a reservoir segment 105b that is proximal to the main segment 105a, and a seal segment 105c that is proximal to the reservoir segment 105b.

In the depicted example, the main segment 105a can include steering components, as described in more detail below. The reservoir segment 105b of the housing 105 comprises the reservoir 132. In this manner the segment 105b of the housing 105 can also be referred to as the “reservoir housing.” The seal segment 105c of the housing 105 comprises the seals 124 (which can also be referred to as a “seal stack”). In this manner, the segment 105d of the housing 105 can also be referred to as the “seal housing.”

The one or more seals 124 of the handle 102 can be configured to fluidly seal the main lumen 122 of the guide sheath 100 from the external environment, while allowing a delivery apparatus (such as any of the prosthetic device delivery apparatuses or implant catheters described herein) to pass therethrough. For example, the one or more seals 124 of the seal segment 105c can be configured to prevent blood from a patient in which the guide sheath 100 is inserted from exiting the guide sheath 100 and prevent air from the environment from entering the guide sheath 100 (e.g., through the inlet port 106), for example, when the delivery apparatus is inserted into and/or removed from the guide sheath 100. The one or more seals 124 can include a variety of types of seals, such as a duckbill seal, a flapper seal, an umbrella valve, a cross-slit valve, a disc valve, a dome valve, or the like.

The reservoir 132 (which can also be referred to as an “air trap”) of the handle 102 is disposed adjacent and distal to the seal segment 105c, and more particularly, distal to the seals 124 of the seal segment 105c. The reservoir 132 can include a wall 134 having an inner surface that defines a reservoir lumen 136 extending the length of the reservoir 132. A tube 116 can be connected to the reservoir 132 via the port 126, and further connects to the reservoir lumen 136 (FIG. 6). The tube 116 can be configured to receive fluid through a lumen thereof. In this way, the tube 116 can be fluidly coupled to the reservoir lumen 132 by the port 126.

The reservoir lumen 136 can be configured to allow air, to the extent any is introduced into the handle 102 as a delivery apparatus is inserted through the seal segment 105c, to accumulate, collect, and/or become trapped within the reservoir lumen 136 of the reservoir 132. Specifically, the reservoir lumen 136 includes an inner diameter ID_{R} that can be greater than an inner diameter ID_{M} of the main lumen 122. In this way, when the handle 102 is positioned in certain orientations (e.g., with the longitudinal axis 112 perpendicular to the direction of gravity, with the port 126 in a vertical direction, etc.), any air introduced through the seal segment 105c gravitates towards the location within the handle 102 having a maximum inner diameter (e.g., the reservoir lumen 136) and can be removed out of the tube 116 via the port 126. In some instances, air that is trapped within reservoir 132 can be removed from the handle 102 to another external location (e.g., a syringe). For example, a syringe may be connected to the tube 116 and used to create a vacuum or negative pressure to remove the air from the reservoir 132 and out of the handle 102 via the port 126 and tube 116.

The inner diameter ID_{R} of the reservoir lumen 136 can be measured between two points on the inner surface of the wall 134 in a radial direction relative to the longitudinal axis 112. Similarly, the inner diameter ID_{M} of the main lumen 122 can be measured between two points on the inner surface of the wall 130 in a radial direction relative to the longitudinal axis 112. In some instances, the main lumen 122 can extend through the reservoir 132 and can be defined, in part, by the inner surface of the wall 134 of the reservoir 132. In these instances, the reservoir lumen 132 is the portion of the main lumen 122 that is defined by the inner surface of the wall 134 (e.g., within the reservoir 132).

As introduced above, the main segment 105a of the handle 102 can include a steering mechanism configured to adjust the curvature of the distal end portion of the shaft 104 (as such, the shaft 104 can be referred to as a steerable shaft). In the illustrated example, the handle 102 includes an adjustment member, such as the illustrated rotatable knob 120. The main segment 105a can house internal flex mechanisms 128 of the guide sheath 100 which are operatively coupled to the rotatable knob 120 (FIG. 6). In some examples, the flex mechanisms 128, and thus the knob 120, can be operatively coupled to the proximal end portion of a pull wire. The pull wire can extend distally from the handle 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Pat. No. 9,339,384, which is incorporated by reference herein.

The main segment 105a of the handle 102 can include a connector 114 (also referred to herein as an “adaptor spine”) disposed adjacent and distal to the reservoir 132. The handle 102 can include one or more gaskets 115 (e.g., o-rings and/or other types of sealing components) disposed between adjacent components to seal any gaps between surfaces of the adjacent components. As shown in FIG. 6, the handle 102 includes a first gasket 115 disposed adjacent and distal to the seal segment 105c and a second gasket 115 disposed adjacent and distal to the reservoir 132. It should be noted that the size of the gaskets 115 shown in FIG. 6 may be exaggerated for the purpose of illustration. In some examples, the width of the gaskets 115 may be smaller than shown in FIG. 6.

FIG. 7 illustrates a delivery apparatus 200 configured to implant a docking device, such as docking device 240 (FIG. 8) described below or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 7, the handle 206 can include knobs 208 and 210 which can be configured to steer or control flexing of the delivery apparatus 200 such as the delivery sheath 204 and/or the sleeve shaft 220 described below.

In certain examples, the delivery apparatus 200 can also include a pusher shaft 212 and a sleeve shaft 220, both of which can extend through an inner lumen of the delivery sheath 204 and have respective proximal end portions extending into the handle assembly 202.

As described below, a distal end portion (also referred to as “distal section”) of the sleeve shaft 220 can be configured to cover (e.g., surround) the docking device 240 (see FIG. 8). For example, the docking device 240 can be retained inside the sleeve shaft 220, which is further retained by a distal end portion 205 of the delivery sheath 204, when navigating through a patient's vasculature.

Additionally, the distal end portion 205 of the delivery sheath 204 can be configured to be steerable. In one example, by rotating a knob (e.g., 208 or 210) on the handle 206, a curvature of the distal end portion 205 can be adjusted so that the distal end portion 205 of the delivery sheath 204 can be oriented in a desired angle. For example, to implant the docking device 240 at the native mitral valve location, the distal end portion 205 of the delivery sheath 204 can be steered in the left atrium so that at least a portion of the sleeve shaft 220 and the docking device 240 retained therein can extend through the native mitral valve annulus at a location adjacent the posteromedial commissure.

In certain examples, the pusher shaft 212 and the sleeve shaft 220 can be coaxial with one another, at least within the delivery sheath 204. In addition, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 240 retained inside the sleeve shaft 220.

After reaching a target implantation site, the docking device 240 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 240 can be pushed out of a distal end 204d of the delivery sheath 204, thus changing from a delivery configuration to a deployed configuration (see FIG. 8). In certain examples, the pusher shaft 212 and the sleeve shaft 220 can be actuated independently of each other.

During delivery, the docking device 240 can be coupled to the delivery apparatus 200 via a release suture (not shown) (or other retrieval line comprising a string, yarn, or other material that can be configured to be tied around the docking device 240 and cut for removal) that extends through the pusher shaft 212. In one specific example, the release suture can extend through the delivery apparatus 200, e.g., through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flush ports (e.g., flush port 232 is shown in FIG. 7) to supply flush fluid to one or more lumens arranged within the delivery apparatus 200 (e.g., annular lumens arranged between coaxial components of the delivery apparatus 200).

Further details on delivery apparatus/catheters/systems (including various examples of the handle assembly) that are configured to deliver a docking device to a target implantation site can be found in International Application No. PCT/US2020/036577 and in U.S. Patent Publication Nos. 2018/0318079 and 2018/0263764, which are all incorporated by reference herein in their entireties.

FIG. 8 illustrates a docking device 240, according to one example. The docking device 240 can, for example, be used as the docking device 52 in a prosthetic valve implantation procedure, as described above with reference to FIGS. 1-4. As depicted in FIG. 8, the docking device in its deployed configuration can be configured to receive and secure a prosthetic valve within the docking device, thereby securing the prosthetic valve at the native valve annulus.

The docking device 240 can comprise a coil 242 and a guard member 244 covering at least a portion of the coil 242. In certain examples, the coil 242 can include a shape memory material (e.g., nickel titanium alloy or “Nitinol”) such that the docking device 240 (and the coil 242) can move from a substantially straight configuration (or delivery configuration) when disposed within the delivery sheath 204 of the delivery apparatus 200 to a helical, deployed configuration after being removed from the delivery sheath 204.

The coil 242 has a proximal end 242p and a distal end 242d (which also respectively define the proximal and distal ends of the docking device 240). When being disposed within the delivery sheath 204 (e.g., during delivery of the docking device 240 into the vasculature of a patient), a body of the coil 242 between the proximal end 242p and distal end 242d can form a generally straight delivery configuration (i.e., without any coiled or looped portions, but can be flexed or bent) so as to maintain a small radial profile when moving through a patient's vasculature. After being removed from the delivery sheath 204 and deployed at an implant position, the coil 242 can move from the delivery configuration to the helical deployed configuration and wrap around native tissue adjacent the implant position. For example, when implanting the docking device at the location of a native valve, the coil 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 8, the coil 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example.

Further details of the docking device and its variants are described in International Application No. PCT/US2021/056150, which is incorporated by reference herein in its entirety.

FIG. 9 illustrates a prosthetic heart valve delivery apparatus 300 (which can also be referred to here as an “implant catheter”) that can be used to implant an expandable prosthetic heart valve, according to one example. In some examples, the delivery apparatus 300 is specifically adapted for use in introducing a prosthetic heart valve into a heart. For example, the delivery apparatus 300 can be used as the prosthetic heart valve delivery apparatus 60 in a prosthetic valve implantation procedure, as described above with reference to FIG. 3A.

The delivery apparatus 300 in the illustrated example of FIG. 9 is a balloon catheter comprising a handle 302 and a steerable, outer shaft 304 extending distally from the handle 302. The delivery apparatus 300 can further comprise an intermediate shaft 306 (which also may be referred to as a balloon shaft) that extends proximally from the handle 302 and distally from the handle 302, the portion extending distally from the handle 302 also extending coaxially through the outer shaft 304. In some examples, the delivery apparatus 300 can further comprise an inner shaft extending distally from the handle 302 coaxially through the intermediate shaft 306 and the outer shaft 304 and proximally from the handle 302 coaxially through the intermediate shaft.

The outer shaft 304 and the intermediate shaft 306 can be configured to translate (e.g., move) longitudinally, along a central longitudinal axis 320 of the delivery apparatus 300, relative to one another to facilitate delivery and positioning of a prosthetic valve at an implantation site in a patient's body.

The intermediate shaft 306 can include a proximal end portion that extends proximally from a proximal end of the handle 302, to an adaptor 312. The adaptor 312 can include a first port 338 configured to receive a guidewire therethrough and a second port 340 configured to receive fluid (e.g., inflation fluid) from a fluid source. The second port 340 can be fluidly coupled to an inner lumen of the intermediate shaft 306.

In some examples, the intermediate shaft 306 can further include a distal end portion that extends distally beyond a distal end of the outer shaft 304 when a distal end of the outer shaft 304 is positioned away from an inflatable balloon 318 of the delivery apparatus 300. A distal end portion of the inner shaft can extend distally beyond the distal end portion of the intermediate shaft 306 toward or to a nose cone 322 at a distal end of the delivery apparatus 300.

In some examples, a distal end of the balloon 318 can be coupled to a distal end of the delivery apparatus 300, such as to the nose cone 322 (as shown in FIG. 9), or to an alternate component at the distal end of the delivery apparatus 300 (e.g., a distal shoulder). An intermediate portion of the balloon 318 can overlay a valve mounting portion 324 of a distal end portion of the delivery apparatus 300 and a distal end portion of the balloon 318 can overly a distal shoulder of the delivery apparatus 300. As shown in FIG. 9, a prosthetic heart valve 350 can be mounted around the balloon 318, at the valve mounting portion 324 of the delivery apparatus 300, in a radially compressed state. The prosthetic heart valve 350 can be configured to be radially expanded by inflation of the balloon 318 at a native valve annulus, as described above with reference to FIG. 3A.

A balloon shoulder assembly of the delivery apparatus 300, which includes the distal shoulder, is configured to maintain the prosthetic heart valve 350 (or other medical device) at a fixed position on the balloon 318 during delivery through the patient's vasculature.

The outer shaft 304 can include a distal tip portion 328 mounted on its distal end. In some examples, the outer shaft 304 and the intermediate shaft 306 can be translated axially relative to one another to position the distal tip portion 328 adjacent to a proximal end of the valve mounting portion 324, when the prosthetic valve 350 is mounted in the radially compressed state on the valve mounting portion 324 (as shown in FIG. 9) and during delivery of the prosthetic valve to the target implantation site. As such, the distal tip portion 328 can be configured to resist movement of the prosthetic valve 350 relative to the balloon 318 proximally, in the axial direction, relative to the balloon 318, when the distal tip portion 328 is arranged adjacent to a proximal side of the valve mounting portion 324.

An annular space can be defined between an outer surface of the inner shaft and an inner surface of the intermediate shaft 306 and can be configured to receive fluid from a fluid source via the second port 340 of the adaptor 312. The annular space can be fluidly coupled to a fluid passageway formed between the outer surface of the distal end portion of the inner shaft and an inner surface of the balloon 318. As such, fluid from the fluid source can flow to the fluid passageway from the annular space to inflate the balloon 318 and radially expand and deploy the prosthetic valve 350.

An inner lumen of the inner shaft can be configured to receive a guidewire therethrough, for navigating the distal end portion of the delivery apparatus 300 to the target implantation site.

The handle 302 can include a steering mechanism configured to adjust the curvature of the distal end portion of the delivery apparatus 300. In the illustrated example, for example, the handle 302 includes an adjustment member, such as the illustrated rotatable knob 360, which in turn is operatively coupled to the proximal end portion of a pull wire. The pull wire can extend distally from the handle 302 through the outer shaft 304 and has a distal end portion affixed to the outer shaft 304 at or near the distal end of the outer shaft 304. Rotating the knob 360 can increase or decrease the tension in the pull wire, thereby adjusting the curvature of the distal end portion of the delivery apparatus 300. Further details on steering or flex mechanisms for the delivery apparatus can be found in U.S. Pat. No. 9,339,384, as previously incorporated by reference above.

The handle 302 can further include an adjustment mechanism 361 including an adjustment member, such as the illustrated rotatable knob 362, and an associated locking mechanism including another adjustment member, configured as a rotatable knob 378. The adjustment mechanism 361 is configured to adjust the axial position of the intermediate shaft 306 relative to the outer shaft 304 (e.g., for fine positioning at the implantation site).

FIG. 10 illustrates the prosthetic valve 350 in a radially expanded position. The prosthetic valve 350 can be used as the prosthetic heart valve 62 in a prosthetic valve implantation procedure, as described above with reference to FIGS. 1-4. The prosthetic valve 350 can include a frame 352 and a plurality of leaflets 354 can be situated at least partially within the frame 352. The prosthetic valve 350 can also include an outer covering 356 situated about the frame 352.

Further details of the prosthetic heart valve and its variants are described in U.S. Pat. No. 11,185,406, which is incorporated by reference herein in its entirety.

As noted above, the delivery apparatus 200 and/or the delivery apparatus 300 can be introduced into a patient's vasculature via a guide catheter, such as the guide sheath 100 of FIGS. 5 and 6. For example, to introduce the delivery apparatus 200 and/or 300 (or an alternate implant catheter or delivery apparatus) into the vasculature of a patient, the shaft 104 of the guide sheath 100 can first be inserted into the patient's vasculature and navigated through the vasculature toward a target implantation site for a medical device or implant. The handle 102 of the guide sheath 100 remains outside the patient and can be accessed by a user (e.g., a physician). The distal end portion 205 of the delivery apparatus 200 and/or the distal end portion of the delivery apparatus 300 (e.g., the nose cone 322 and radially compressed prosthetic heart valve 350 of the delivery apparatus 300) can then be inserted into the inlet port 106 of the handle 102 of the guide sheath 100. The distal end portion of the delivery apparatus 200 and/or 300 is then pushed through the seal segment 105c (e.g., through the seals 124) and into the reservoir 132 of the guide sheath 100. The delivery apparatus 200 and/or 300 can then continue to be pushed through the inner lumen of the shaft 104, toward the implantation site.

As mentioned above, it is possible for air to be introduced within the system when the delivery apparatus 200 and/or 300 is inserted into the guide sheath 100 in some examples. To prevent any air that may have entered the system from advancing distally through the system, the inventors herein have realized that it is advantageous to provide a reservoir of fluid which is configured to trap the air within the reservoir, such that the trapped air may be removed and/or aspirated out of the system from the reservoir.

Referring again to FIGS. 5 and 6, the reservoir 132 of the guide sheath 100 can be configured to prevent air (to the extent any has entered the system) from advancing further (or distally) into the system (e.g., distal to the reservoir 132, etc.). Specifically, as described above, the reservoir 132 can be located within the reservoir segment 105b of the handle 102 and can be positioned distal to the seal segment 105c. As noted above, the inner diameter ID_{R} of the reservoir lumen 136 can be larger than the inner diameter ID_{M} of the main lumen 122. Because of the difference in inner diameter, any air that is introduced by way of a delivery apparatus (e.g., delivery apparatus 200 and/or 300) being inserted through the seal segment 105c can be trapped within the reservoir and prevented from traveling distally through the guide sheath 100. Any air trapped within the reservoir 132 may be aspirated or removed through the tube 116 via the port 126 (e.g., using a syringe).

As shown in FIG. 6, the inner diameter ID_{R} of the reservoir lumen 136 is a constant value from a proximal end of the reservoir 132 to a distal end of the reservoir 132 (e.g., the reservoir lumen 136 is a straight bore, etc.). It should be appreciated that in other instances, the inner diameter ID_{R} of the reservoir lumen 136 can vary (e.g., increase in a proximal direction, etc.) such that the reservoir lumen 136 can include one or more tapers (e.g., a tapered or frustoconical portion, etc.), steps, or the like, as discussed in more detail below (see also FIGS. 11A-B).

The reservoir lumen 132 can have a circular cross-section. In some instances, the cross-section of the reservoir lumen 132 can include other shapes, including but not limited to round shapes such as ovals, ellipses, etc. and/or polygonal shapes such as squares, rectangles, etc. For example, in some instances, the reservoir lumen 132 may be generally cylindrical and include an axially extending slot disposed in a radially spaced position from the longitudinal axis 112. Further, in some instances, the cross-section of the reservoir lumen 132 can be asymmetrical (e.g., such that a central longitudinal axis of the reservoir lumen 132 is not coaxial with the longitudinal axis 112 of the guide sheath 100). It should be appreciated that in instances where the cross-section of the reservoir lumen 132 is non-circular, the inner diameter ID_{R} refers to a greatest measurement (e.g., length, width, diagonal, etc.) of the cross-section (e.g., a measurement between two points on the inner surface of the wall 134 in a radial direction relative to the longitudinal axis 112, etc.).

In some examples, the reservoir 132 can be a transparent or translucent material (e.g., a clear polycarbonate material, etc.) to enable a user of the guide sheath to visually inspect whether any air has entered the system (e.g., after insertion of a delivery apparatus, etc.).

The port 126 can be perpendicular to the longitudinal axis 112, as shown in FIG. 6. In some examples, the port 126 can be positioned at a different angle with respect to the longitudinal axis 112 (e.g., angled toward the proximal end 132p of the reservoir 132, etc.).

FIG. 11A shows an example of a reservoir 432 (or air trap) of guide sheath 400 that includes a reservoir lumen 436 having a taper 438 (also referred to as a “tapered portion” or “frustoconical portion”). Similar to reservoir 132, the reservoir 432 of guide sheath 400 is configured to prevent air (to the extent any has entered the system) from advancing further (or distally) into the system, whereby any air trapped within the reservoir 432 may be removed through a tube (e.g., tube 116 shown in FIG. 5) via a port 426. The guide sheath 400 can be similar to the guide sheath 100 of FIGS. 5-6. Thus, common components between the guide sheath 400 and the guide sheath 100 are labeled similarly in FIG. 11A and not redescribed below, for the sake of brevity.

In the illustrated example, the reservoir lumen 436 can include an inner diameter that varies from a maximum inner diameter ID_{R} to a minimum inner diameter that is equal to an inner diameter ID_{M} of the main lumen 422. As shown, the reservoir lumen 436 can include a taper 438 (e.g., a gradual narrowing, a uniform change in inner diameter, etc.) from the inner diameter ID_{R} to the inner diameter ID_{M} of the main lumen 422 in a distal direction. Specifically, the reservoir 432 can include a band 440 (also referred to as a “cylindrical portion”) where the inner diameter of the reservoir lumen 436 is at the maximum inner diameter ID_{R} and the taper 438 is distal to the band 440 and extends to a distal end of the reservoir 432. In this way, the reservoir lumen 436 is generally shaped as a funnel. The reservoir lumen 436 can also include a step 442 (e.g., an abrupt change, a right angle, etc.) from the inner diameter ID_{R} to the inner diameter ID_{M} of the main lumen 422 in a proximal direction. Specifically, the step 442 can be proximal to the band 440 (e.g., adjacent to a proximal end of the reservoir 432). The inner diameter of the reservoir lumen 436 at the step 442 can be equal to the inner diameter ID_{M} of the main lumen 422.

It should be appreciated that in some examples the reservoir 432 can include alternate configurations of tapers, bands, and/or steps. For example, in some instances, the band 440 can be omitted, such that the taper 438 is directly adjacent and distal to the step 442. Further, in some examples, the reservoir 432 can only include the taper 438, such that the inner diameter of the reservoir lumen 436 uniformly increases from the inner diameter ID_{M} of the main lumen 422 at a distal end of the reservoir 432 to the maximum inner diameter ID_{R} at the proximal end of the reservoir 432. It should be appreciated that in some examples, the taper 438 does not extend to the distal end of the reservoir 432. In these examples, the reservoir lumen 426 can include a portion at the distal end of the reservoir 432 having an inner diameter equal to the inner diameter ID_{M} of the main lumen 122 that is distal to the taper 438.

As described above, the reservoir 432 of the guide sheath 400 includes a port 426 which fluidly couples the reservoir lumen 436 (and the main lumen 402) with a flush port 416 of the handle 102. In the illustrated example, the port 426 can be positioned at the band 440 and in fluid connection with the reservoir lumen 436. The port 426 can be perpendicular to the longitudinal axis 412, as shown in FIG. 11A. In some examples, the port 426 can be positioned at a different angle with respect to the longitudinal axis 412 (e.g., angled toward the proximal end of the reservoir 432, etc.).

In some examples, the reservoir 432 can be a transparent or translucent material (e.g., a clear polycarbonate material, etc.) to enable a user of the guide sheath to visually inspect whether any air has entered the system (e.g., after insertion of a delivery apparatus, etc.).

FIG. 11B shows another example of a reservoir 532 (or air trap) of a guide sheath 500 that includes a reservoir lumen 536 configured to trap any air that may have entered the handle 502 of the guide sheath 500, prevent such air from moving distally through the handle 502, and/or allow removal of the air through the tube 516. The guide sheath 500 can be similar to the guide sheath 100 of FIGS. 5 and 6 and/or to the guide sheath 400 of FIG. 11A. Thus, common components between the guide sheath 500, the guide sheath 100, and/or the guide sheath 400 are labeled similarly in FIG. 11B and not redescribed below, for the sake of brevity.

In the illustrated example, the reservoir lumen 536 can include an inner diameter that varies from a maximum inner diameter ID_{R} to a minimum inner diameter that is equal to an inner diameter ID_{M} of the main lumen 522. As shown, the reservoir lumen 536 can include a first (or distal) taper 544 (e.g., a gradual narrowing, a uniform change in inner diameter, etc.) from the inner diameter ID_{R} to the inner diameter ID_{M} of the main lumen 422 in a distal direction. The reservoir lumen 536 can also include a second (or proximal) taper 546 (e.g., a gradual narrowing, a uniform change in inner diameter, etc.) from the inner diameter ID_{R} to the inner diameter ID_{M} of the main lumen 422 in a proximal direction. Specifically, the reservoir 532 can include a band 548 (also referred to as a “cylindrical portion”) where the inner diameter of the reservoir lumen 536 is at the maximum inner diameter ID_{R}, the first taper 544 is distal to the band 548 and extends to a distal end of the reservoir 532 and the second taper 546 is proximal to the band 548 and extends to a proximal end of the reservoir 532. In this way, the reservoir lumen 536 generally includes an intermediate expanded (or cylindrical) portion that is disposed between two tapered (or frustoconical) portions. The inner diameter of the reservoir lumen 436 at the step 442 can be equal to the inner diameter ID_{M} of the main lumen 422. It should be appreciated that in some instances, the band 548 can be omitted, such that the first taper 544 is directly adjacent and distal to the second taper 546. Additionally, in some examples, the first taper 544 and/or the second taper 546 may be replaced with steps (e.g., similar to step 442).

As described above, the reservoir 532 of the guide sheath 500 includes a port 526 which fluidly couples the reservoir lumen 536 (and the main lumen 502) with a tube 516. In the illustrated example, the port 526 can be positioned at the second taper 546 and in fluid connection with the reservoir lumen 536. It should be appreciated that in other instances, the port 526 can be positioned at other portions of the reservoir lumen 536 (e.g., the first taper 544, the band 548, etc.). The port 526 can be perpendicular to the longitudinal axis 512, as shown in FIG. 11B. In some examples, the port 526 can be positioned at a different angle with respect to the longitudinal axis 512 (e.g., angled toward the proximal end of the reservoir 532, etc.).

In some examples, the reservoir 532 can be a transparent or translucent material (e.g., a clear polycarbonate material, etc.) to enable a user of the guide sheath to visually inspect whether any air has entered the system (e.g., after insertion of a delivery apparatus, etc.).

FIG. 12 shows an example of a reservoir 632 (or air trap) of a handle of a guide sheath (e.g., similar to guide sheath 100, 400, 500) that is configured to prevent air (to the extent any has entered the system) from advancing further (or distally) into the system, whereby any air trapped within the reservoir 632 may be removed through a flush port via a flush lumen 626. The reservoir 632 can be similar to the reservoir 132 of FIGS. 5-6, the reservoir 432 of FIG. 11A, and/or the reservoir 532 of FIG. 11B. Thus, common components between the reservoir 632 and the reservoirs 132, 432, and/or 532 are labeled similarly in FIG. 12 and not redescribed below, for the sake of brevity. Additionally, components described below in connection with the reservoir 632 may also be included in the reservoirs 132, 432, and/or 532.

The reservoir 632 can include an inlet 650 at a proximal end of the reservoir 632 and an outlet 652 at a distal end of the reservoir 632. The inlet 650 can provide an entryway into the reservoir lumen 636 to allow insertion of a delivery apparatus (such as any of the prosthetic device delivery apparatuses or implant catheters described herein) into the reservoir lumen 636. The inlet 650 (or the proximal end) of the reservoir 632 is configured for attachment to a seal housing assembly of a handle (e.g., seal segment 105c, etc.) and the outlet 652 (or distal end) of the reservoir 632 is configured for attachment to a main body portion of a handle (e.g., main body portion 118 via connector 114). Specifically, the inlet 650 and the outlet 652 can include a plurality of attachment points 654 (e.g., holes, threaded holes, etc.) which are configured to receive fasteners (e.g., screws, etc.) (not shown) to enable the reservoir 632 to be coupled at an intermediate position within a handle (e.g., distal to a seal housing assembly, etc.).

In the illustrated example, the flush lumen 626 is disposed near the proximal end of the reservoir 632. It should be appreciated that in other examples, the flush lumen 626 may be disposed in other positions on the reservoir 632 (e.g., near the distal end of the reservoir 632, at a maximum inner diameter of the reservoir lumen 636, etc.).

In this way, the reservoirs (or air traps) of the delivery apparatuses described above with reference to FIGS. 5-6 and 11A-12 can provide a location for air (to the extent any is introduced into the delivery apparatuses) to accumulate and/or become trapped as implant catheters and other devices are being navigated through the delivery apparatuses. In some examples, air can enter a delivery apparatus as an implant catheter is inserted into a seal housing assembly at a proximal end of a handle of the delivery apparatus. The air can then accumulate and become trapped (e.g., prevented from travelling distally through the handle, etc.) within a reservoir of the handle that is positioned adjacent and distal to the seal housing assembly. As a result of the air becoming trapped within the reservoir, the air can be removed and/or aspirated from the reservoir via a flush port. In some examples, this can increase an efficiency of a prosthetic device implantation procedure.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle comprising: a proximal segment including one or more fluid seals mounted within the proximal segment, the fluid seals configured to allow insertion of a device into the handle and prevent fluid flow past the fluid seals; an intermediate segment disposed adjacent and distal to the proximal segment, the intermediate segment including an inner surface defining a first lumen, wherein the first lumen has a first inner diameter, wherein the first lumen includes an inlet and an outlet; and a distal segment disposed adjacent and distal to the intermediate segment; and a shaft extending distally from the handle, the shaft including a distal end, a proximal end, and a second lumen extending between the distal end and the proximal end of the shaft, wherein the proximal end of the shaft is disposed within the distal segment of the handle and is coupled to the outlet of the first lumen, wherein the second lumen includes a second inner diameter that is smaller than the first inner diameter of the first lumen.

Example 2. The delivery apparatus of any example herein, particularly example 1, wherein the first lumen and the second lumen are coaxial.

Example 3. The delivery apparatus of any example herein, particularly either example 1 or example 2, wherein the inlet of the first lumen is coupled to the proximal segment of the handle.

Example 4. The delivery apparatus of any example herein, particularly any one of examples 1-3, wherein the inner surface includes at least one taper.

Example 5. The delivery apparatus of any example herein, particularly any one of examples 1-4, wherein the inner surface includes a band having a constant inner diameter equal to the first inner diameter.

Example 6. The delivery apparatus of any example herein, particularly example 5, wherein the band extends from the inlet of the first lumen to the outlet of the first lumen.

Example 7. The delivery apparatus of any example herein, particularly example 5, wherein the band includes a distal end and a proximal end, wherein the inner surface is tapered from the distal end of the band to the outlet of the first lumen.

Example 8. The delivery apparatus of any example herein, particularly example 7, wherein the inner surface includes a step disposed between the inlet of the first lumen and the proximal end of the band.

Example 9. The delivery apparatus of any example herein, particularly example 7, wherein the inner surface is tapered from the proximal end of the band to the inlet of the first lumen.

Example 10. The delivery apparatus of any example herein, particularly any one of examples 1-9, further comprising a flush port coupled to the intermediate segment, the flush port fluidly coupled to the first lumen via a flush lumen.

Example 11. The delivery apparatus of any example herein, particularly any one of examples 1-10, wherein an inner diameter of the outlet of the first lumen is equal to the second inner diameter.

Example 12. The delivery apparatus of any example herein, particularly any one of examples 1-11, wherein an inner diameter of the inlet of the first lumen is equal to the second inner diameter.

Example 13. The delivery apparatus of any example herein, particularly any one of examples 1-12, wherein the intermediate segment is transparent or translucent.

Example 14. A delivery apparatus comprising: a seal housing assembly including a plurality of fluid seals, the plurality of fluid seals configured to allow insertion of a device into the delivery apparatus and prevent fluid flow past the fluid seals; a reservoir coupled to and extending distally from the seal housing assembly, the reservoir including a reservoir lumen having a first inner diameter and a flush lumen in fluid communication with the reservoir lumen, wherein the reservoir lumen includes an inlet coupled to the seal housing assembly and an outlet; and a shaft coupled to and extending distally from the outlet of the reservoir, the shaft including a shaft lumen in fluid communication with the reservoir lumen, and wherein the shaft lumen includes a second inner diameter that is smaller than the first inner diameter of the reservoir lumen.

Example 15. The delivery apparatus of any example herein, particularly example 14, further comprising a flush port coupled to the reservoir, the flush port fluidly coupled to the flush lumen.

Example 16. The delivery apparatus of any example herein, particularly either example 14 or example 15, wherein the reservoir lumen includes at least one cylindrical portion.

Example 17. The delivery apparatus of any example herein, particularly example 16, wherein the flush lumen is connected to the cylindrical portion of the reservoir lumen.

Example 18. The delivery apparatus of any example herein, particularly any one of examples 14-17, wherein the reservoir lumen includes at least one frustoconical portion.

Example 19. The delivery apparatus of any example herein, particularly example 18, wherein the flush lumen is connected to the frustoconical portion of the reservoir lumen.

Example 20. The delivery apparatus of any example herein, particularly any one of examples 14-19, wherein the flush lumen is positioned adjacent to a proximal end of the reservoir.

Example 21. The delivery apparatus of any example herein, particularly any one of examples 14-20, wherein the flush lumen is perpendicular to a longitudinal axis of the reservoir.

Example 22. The delivery apparatus of any example herein, particularly any one of examples 14-21, wherein the reservoir is transparent or translucent.

Example 23. The delivery apparatus of any example herein, particularly any one of examples 14-22, further comprising a gasket disposed between the reservoir and the seal housing assembly.

Example 24. A method for implanting a prosthetic medical device, comprising: inserting a shaft of a guide catheter into a vessel of a patient; inserting a distal end portion of a first implant catheter into a proximal end of a handle of the guide catheter and pushing the distal end portion of the first implant catheter through a reservoir of the handle of the guide catheter and then through a main lumen of the shaft of the guide catheter toward a target implantation site for a prosthetic medical device mounted on the distal end portion of the first implant catheter, the reservoir having an inner diameter that is larger than an inner diameter of the main lumen; and after inserting the distal end portion of the first implant catheter, removing fluid and/or air out of the reservoir through a tube that is fluidly coupled with the reservoir via a port of the reservoir.

Example 25. The method of any example herein, particularly example 24, wherein the shaft of the guide catheter extends into the handle of the guide catheter which remains exterior to the patient while a portion of the shaft extending distally from the handle is disposed within the vessel.

Example 26. The method of any example herein, particularly either example 24 or example 25, wherein the flush port is disposed distal to one or more fluid seals of the handle that are disposed adjacent to the proximal end of the guide catheter.

Example 27. The method of any example herein, particularly any one of examples 24-26, further comprising implanting the prosthetic medical device at the target implantation site, removing the first implant catheter from the guide catheter, and inserting a second implant catheter into the guide catheter and pushing the second implant catheter through the reservoir and through the main lumen toward the target implantation site.

Example 28. The method of any example herein, particularly example 27, further comprising after inserting the second implant catheter, aspirating fluid and/or air out of the reservoir through the flush port.

Example 29. The method of any example herein, particularly either example 27 or example 28, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 30. A delivery apparatus comprising: a shaft including a first lumen having a first inner diameter; and a handle including an air trap portion connected to a proximal end of the shaft and a seal stack portion adjacent and proximal to the air trap portion, the air trap portion including a second lumen coaxial with the first lumen, the second lumen having a second inner diameter that is larger than the first inner diameter, wherein the seal stack portion includes one or more fluid seals mounted within the seal stack portion and configured to allow insertion of a device therethrough.

Example 31. The delivery apparatus of any example herein, particularly example 30, wherein the second lumen is a straight bore.

Example 32. The delivery apparatus of any example herein, particularly example 30, wherein the second inner diameter of the second lumen increases uniformly in a proximal direction, from a distal end of the second lumen to an intermediate location of the second lumen.

Example 33. The delivery apparatus of any example herein, particularly example 30, wherein the second inner diameter of the second lumen decreases uniformly in a proximal direction, from an intermediate location to a proximal end of the second lumen.

Example 34. The delivery apparatus of any example herein, particularly any one of examples 30-33, further comprising a flush port coupled to the air trap portion of the handle, wherein the flush port is fluidly coupled to the second lumen via a flush lumen.

Example 35. The delivery apparatus of any example herein, particularly any one of examples 30-34, further comprising a gasket positioned between the air trap portion and the seal stack portion.

Example 36. The delivery apparatus of any example herein, particularly any one of examples 30-35, wherein the air trap portion is coupled to the seal stack portion with fasteners.

Example 37. The delivery apparatus of any example herein, particularly any one of examples 30-36, wherein the air trap portion is transparent.

Example 38. The delivery apparatus of any example herein, particularly any one of examples 30-37, wherein a proximal end of the shaft is disposed within the handle.

Example 39. A delivery assembly comprising: an implant catheter; and a guide catheter comprising: a shaft having a distal end and a proximal end, the shaft including a main lumen configured to receive a portion of the implant catheter therethrough, the main lumen having a first inner diameter; and a handle including a reservoir coupled to the proximal end of the shaft and a seal housing assembly adjacent and proximal to the reservoir, the reservoir including a reservoir lumen in fluid communication with the main lumen, the reservoir lumen having a second inner diameter that is larger than the first inner diameter, wherein the seal housing assembly includes one or more fluid seals mounted within the seal housing assembly and configured to allow insertion of the implant catheter therethrough.

Example 40. The delivery assembly of any example herein, particularly example 39, wherein the reservoir lumen is coaxial with the main lumen.

Example 41. The delivery assembly of any example herein, particularly either example 39 or example 40, wherein the proximal end of the shaft is disposed within the handle.

Example 42. The delivery assembly of any example herein, particularly any one of examples 39-41, wherein the implant catheter is one of a dock delivery catheter and a prosthetic heart valve delivery catheter.

Example 43. The delivery assembly of any example herein, particularly any one of examples 39-42, further comprising a prosthetic medical device coupled to the implant catheter.

Example 44. The delivery assembly of any example herein, particularly example 43, wherein the prosthetic medical device is one of a docking device and a prosthetic heart valve.

Example 45. The delivery assembly of any example herein, particularly any one of examples 39-44, wherein the reservoir lumen includes a cylindrical portion.

Example 46. The delivery assembly of any example herein, particularly example 45, wherein the reservoir lumen includes a distal tapered portion adjacent and distal to the cylindrical portion.

Example 47. The delivery assembly of any example herein, particularly example 46, wherein the reservoir lumen includes a step adjacent and proximal to the cylindrical portion.

Example 48. The delivery assembly of any example herein, particularly example 46, wherein the reservoir lumen incudes a proximal tapered portion adjacent and proximal to the cylindrical portion.

Example 49. A delivery apparatus comprising: a handle having a proximal end and a distal end, the handle comprising: a seal housing assembly at the proximal end of the handle, the seal housing assembly including one or more fluid seals mounted within the seal housing assembly, the fluid seals configured to allow insertion of a device into the handle and prevent fluid flow past the fluid seals; an outer housing at the distal end of the handle; and a reservoir having an inlet and an outlet, the inlet coupled to the seal housing assembly and the outlet coupled to the outer housing, the reservoir including a first lumen extending from the inlet to the outlet, the first lumen having a first inner diameter; and a shaft having a proximal end and a distal end, wherein the proximal end of the shaft is disposed within the outer housing and is coupled to the outlet of the reservoir, wherein the second lumen includes a second inner diameter that is smaller than the first inner diameter of the first lumen.

Example 50. The delivery apparatus of any example herein, particularly example 49, wherein the first lumen and the second lumen are coaxial.

Example 51. The delivery apparatus of any example herein, particularly either example 49 or example 50, wherein the first lumen includes at least one taper and/or at least one step.

Example 52. The delivery apparatus of any example herein, particularly any one of examples 49-51, further comprising a flush port connected to the handle, the flush port distal to the fluid seals.

Example 53. The delivery apparatus of any example herein, particularly any one of examples 49-52, wherein the reservoir is a transparent or translucent material.

The features described herein with regard to any example can be combined with other features described in any one or more of the other examples, unless otherwise stated. For example, any one or more of the features of one guide catheter can be combined with any one or more features of another guide catheter. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus comprising:
a handle comprising:
a proximal segment including one or more fluid seals mounted within the proximal segment, the fluid seals configured to allow insertion of a device into the handle and prevent fluid flow past the fluid seals;
an intermediate segment disposed adjacent and distal to the proximal segment, the intermediate segment including an inner surface defining a first lumen, wherein the first lumen has a first inner diameter, wherein the first lumen includes an inlet and an outlet; and
a distal segment disposed adjacent and distal to the intermediate segment; and
a shaft extending distally from the handle, the shaft including a distal end, a proximal end, and a second lumen extending between the distal end and the proximal end of the shaft, wherein the proximal end of the shaft is disposed within the distal segment of the handle and is coupled to the outlet of the first lumen, wherein the second lumen includes a second inner diameter that is smaller than the first inner diameter of the first lumen.

2. The delivery apparatus of claim 1, wherein the first lumen and the second lumen are coaxial.

3. The delivery apparatus of claim 1, wherein the inlet of the first lumen is coupled to the proximal segment of the handle.

4. The delivery apparatus of claim 1, wherein the inner surface includes at least one taper.

5. The delivery apparatus of claim 1, wherein the inner surface includes a band having a constant inner diameter equal to the first inner diameter.

6. The delivery apparatus of claim 5, wherein the band extends from the inlet of the first lumen to the outlet of the first lumen.

7. The delivery apparatus of claim 5, wherein the band includes a distal end and a proximal end, wherein the inner surface is tapered from the distal end of the band to the outlet of the first lumen.

8. The delivery apparatus of claim 7, wherein the inner surface includes a step disposed between the inlet of the first lumen and the proximal end of the band.

9. The delivery apparatus of claim 7, wherein the inner surface is tapered from the proximal end of the band to the inlet of the first lumen.

10. The delivery apparatus of claim 1, further comprising a flush port coupled to the intermediate segment, the flush port fluidly coupled to the first lumen via a flush lumen.

11. The delivery apparatus of claim 1, wherein an inner diameter of the outlet of the first lumen is equal to the second inner diameter.

12. The delivery apparatus of claim 1, wherein an inner diameter of the inlet of the first lumen is equal to the second inner diameter.

13. A delivery assembly comprising:
an implant catheter; and
a guide catheter comprising:
a shaft having a distal end and a proximal end, the shaft including a main lumen configured to receive a portion of the implant catheter therethrough, the main lumen having a first inner diameter; and
a handle including a reservoir coupled to the proximal end of the shaft and a seal housing assembly adjacent and proximal to the reservoir, the reservoir including a reservoir lumen in fluid communication with the main lumen, the reservoir lumen having a second inner diameter that is larger than the first inner diameter, wherein the seal housing assembly includes one or more fluid seals mounted within the seal housing assembly and configured to allow insertion of the implant catheter therethrough.

14. The delivery assembly of claim 13, wherein the reservoir lumen is coaxial with the main lumen.

15. The delivery assembly of claim 13, wherein the proximal end of the shaft is disposed within the handle.

16. The delivery assembly of claim 13, further comprising a prosthetic medical device coupled to the implant catheter.

17. The delivery assembly of claim 16, wherein the prosthetic medical device is one of a docking device and a prosthetic heart valve.

18. A method for implanting a prosthetic medical device, comprising:
inserting a shaft of a guide catheter into a vessel of a patient;
inserting a distal end portion of a first implant catheter into a proximal end of a handle of the guide catheter and pushing the distal end portion of the first implant catheter through a reservoir of the handle of the guide catheter and then through a main lumen of the shaft of the guide catheter toward a target implantation site for a prosthetic medical device mounted on the distal end portion of the first implant catheter, the reservoir having an inner diameter that is larger than an inner diameter of the main lumen; and
after inserting the distal end portion of the first implant catheter, removing fluid and/or air out of the reservoir through a tube that is fluidly coupled with the reservoir via a port of the reservoir.

19. The method of claim 18, further comprising implanting the prosthetic medical device at the target implantation site, removing the first implant catheter from the guide catheter, and inserting a second implant catheter into the guide catheter and pushing the second implant catheter through the reservoir and through the main lumen toward the target implantation site.

20. The method of claim 19, further comprising after inserting the second implant catheter, aspirating fluid and/or air out of the reservoir through the port.
